(19) United States
(12) Patent Application Publication
(10) Pub. No.: US 2007/0161120 A1
(43) Pub. Date: Jul. 12, 2007

(54) Ceruloplasmin Assay

(76) Inventor: Viorica Lopez-Avila, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/330,003

(22) Filed: Jan. 10, 2006

(51) Int. Cl. G01N 33/558

(52) U.S. Cl. 436/514

(57) ABSTRACT

The present invention provides a method of evaluating a sample for ceruloplasmin. The method includes contacting the sample with a multiple immunoaffinity removal medium to result in an unbound fraction which includes the ceruloplasmin. The unbound fraction is subjected to a separation method to provide an aliquot which includes purified ceruloplasmin. The aliquot is then analyzed using ICP-MS to provide a copper ion specific signal; and the sample is evaluated for ceruloplasmin based on the copper ion specific signal.

### BACKGROUND

Ceruloplasmin is the primary copper containing protein in plasma. Ceruloplasmin's main clinical importance is in the diagnosis of Wilson's disease, which presents reduced serum ceruloplasmin concentration and increased dialysable copper concentration. Low serum ceruloplasmin levels also are found in malnutrition, malabsorption, nephrosis and severe liver disease (cirrhosis).

The serum ceruloplasmin concentration rises significantly during acute inflammation due to causes including surgery, myocardial infarction, infections and tumors. High levels of ceruloplasmin in plasma were associated with higher incidence of myocardial infarction (Amer. J. Epidemiology 1992, 136, 1082). Ceruloplasmin concentration typically increases during pregnancy or during the use of oral contraception. Increased ceruloplasmin levels are also associated with diseases of the reticuloendothelial system, such as Hodgkin's disease.

Current methods of determining ceruloplasmin levels include immunoassays, such as sandwich immunoassays; immunoturbidimetric assays; enzyme activity assays; or assays measuring copper concentration. Most of the copper ion in plasma is bound by ceruloplasmin. For example, one report indicates 65% of copper ion is bound to ceruloplasmin, 15% to albumin, 15% to transcuprein and 5% to low MW proteins (Analusis Magazine 1998, 26, M68); and another report indicates 71% of copper ion is bound to ceruloplasmin, 19% to albumin, 7% to transcuprein, and 2% to aminoacids (Eur. J. Clin. Invest 1988, 18, 555). Total copper concentration provides only an approximate measure of ceruloplasmin concentration. As an alternative to the total copper ion measurement, Inagaki et al. (Analyst. (2000) 125(1):197-203) describe using a chelating resin to remove loosely bound copper and zinc ions from serum samples prior to analysis of the samples by inductively coupled plasma-mass spectrometry (ICP-MS).

Immunoassays for ceruloplasmin are described in: U.S. Pat. No. 5,491,066, describing assay methods employing an anti-human ceruloplasmin monoclonal antibody. U.S. Pat. No. 6,806,044 describes another immunoassay for measuring ceruloplasmin concentration. An immunoturbidimetric assay for measuring ceruloplasmin concentration is commercially supplied by Olympus Life and Material Science (cat. no. OSR6164).

Despite the availability of assays for ceruloplasmin such as those described above, there remains a need for new assays for ceruloplasmin.

### SUMMARY OF THE INVENTION

The present invention provides a method of evaluating a sample for ceruloplasmin. The sample will typically include serum abundant proteins and ceruloplasmin. The method includes contacting the sample with a multiple immunoaffinity removal medium under conditions sufficient to provide for binding of the serum abundant proteins to the multiple immunoaffinity removal medium to result in an unbound fraction which includes the ceruloplasmin. The unbound fraction is then subjected to a separation method to provide an aliquot which includes purified ceruloplasmin. The aliquot is then analyzed using ICP-MS to provide a copper ion specific signal; and the sample is evaluated for ceruloplasmin based on the copper ion specific signal.

Additional objects, advantages, and novel features of this invention shall be set forth in part in the descriptions and examples that follow and in part will become apparent to those skilled in the art upon examination of the specification or may be learned by the practice of the invention. The objects and advantages of the invention may be realized and attained by means of the instruments, combinations, compositions and methods particularly pointed out in the specification and in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 schematically illustrates an embodiment of a method according to the present invention.

FIG. 2A illustrates an elution profile.

FIG. 2B illustrates an elution profile.

FIG. 2C illustrates an elution profile.

FIG. 3A illustrates an elution profile.

FIG. 3B illustrates an elution profile.

FIG. 3C illustrates an elution profile.

FIG. 4 illustrates a standard curve of ceruloplasmin concentration.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that, as used in the specification and the appended claims, the singular forms “a,” “an” and “the” include plural referents unless the context clearly dictates otherwise. Thus, for example, reference to “a protein” includes a plurality of proteins. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. In this specification and in the claims that follow, reference will be made to a number of terms that shall be defined to have the following meanings unless a contrary intention is apparent.

“Optional” or “optionally” means that the subsequently described circumstance may or may not occur, so that the description includes instances where the circumstance occurs and instances where it does not. For example, the phrase “optionally recovered” means that a non-hydrogen substituent may or may not be present, and, thus, the description includes structures wherein a non-hydrogen substituent is present and structures wherein a non-hydrogen substituent is not present.

The term “sample” as used herein relates to a material or mixture of materials, typically, although not necessarily, in fluid form, containing one or more components of interest. Typically, the sample is obtained from a biological source, such as a plant or animal source, or eukaryotic cell culture, yeast culture, bacterial culture, or from any other biological source. The sample will typically be obtained from blood, e.g. blood plasma or blood serum.

“Isolated” or “purified” generally refers to isolation of a substance (compound, polynucleotide, protein, polypeptide) such that the substance comprises a significant percent (e.g., greater than 1%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well known in the art and include, for example, ion-exchange chromatography, affinity chromatography and sedimentation according to density. Generally, a substance is purified when it exists in a sample in an amount, relative to other components of the sample that is not found naturally. For the purpose of the percentages related in this paragraph, the solvent is omitted (e.g. the percentages are based on dry weight), and the percentages are calculated as the dry weight of the purified substance divided by the dry weight of all of the substances in the pre-purified sample.

The terms “specific binding”, “specifically bind”, or other like terms, refers to the ability of a binding agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the binding constant of a binding agent and analyte is greater than 10⁶ M−1, greater than 10⁷ M−1, greater than 10⁸ M−1, greater than 10⁹ M−1, greater than 10¹⁰ M−1, usually up to about 10¹² M−1, or even up to about 10¹⁵ M−1. In particular embodiments herein, the binding agents may be antibodies which specifically bind to their target antigens, such as particular serum abundant proteins. “Specific binding conditions” are conditions sufficient to allow a binding agent to preferentially bind to a particular analyte.

“Serum abundant proteins” are those proteins which are typically most prevalent of the proteins present in blood serum of vertebrate organisms, e.g. mammalian, avian, amphibian, or reptilian organisms. Typical examples of serum abundant proteins include albumin, IgG, IgM, IgA, fibrinogen, alpha-2 macroglobulin, transferrin, alpha-1 antitrypsin, haptoglobin, alpha-1 acid glycoprotein, apolipoprotein A-I, and apolipoprotein A-II.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. As another example, if data is used to provide a result, the result is obtained by a process that involves accessing, formatting, transforming, evaluating, and/or analyzing the data, e.g. by applying one or more algorithms to the data.

Accordingly, the present invention provides a method of evaluating a sample for ceruloplasmin. The sample will typically include serum abundant proteins and ceruloplasmin. The method includes contacting the sample with a multiple immunoaffinity removal medium under conditions sufficient to provide for binding of the serum abundant proteins to the multiple immunoaffinity removal medium to result in an unbound fraction which includes the ceruloplasmin. The unbound fraction is then subjected to a separation method to provide an aliquot which includes purified ceruloplasmin. The aliquot is then analyzed using ICP-MS to provide a copper ion specific signal; and the sample is evaluated for ceruloplasmin based on the copper ion specific signal.

The multiple immunoaffinity removal medium specifically binds to at least three, e.g. at least four, at least five, at least six (or more) proteins selected from the group consisting of albumin, IgG, IgM, IgA, fibrinogen, alpha-2 macroglobulin, transferrin, alpha-1 antitrypsin, haptoglobin, alpha-1 acid glycoprotein, apolipoprotein A-I, and apolipoprotein A-II. In typical embodiments, the multiple immunoaffinity removal medium comprises an insoluble substrate having antibodies specific for the indicated serum abundant proteins bound thereto. Insoluble substrates for use in immunoaffinity applications are known in the art and are commercially available. Examples of multiple immunoaffinity removal media which are presently commercially available include PROTEOMELAB IgY spin column solution (Beckman Coulter, Fullerton, Calif.), SEPPRO immunoaffinity protein partitioning system using PROTEOMELAB-IgY immunoaffinity columns (GenWay Biotech, San Diego, Calif.); MARS Multiple Affinity Removal System column (Agilent Technologies, Palo Alto, Calif.). These media may be used according to published protocols for removing serum abundant proteins from serum-derived samples prior to further analysis.

In use, the mixed immunoaffinity removal medium is contacted with the sample, which includes ceruloplasmin as well as serum abundant proteins. This contacting is done under conditions sufficient to result in at least some of the serum abundant proteins being specifically bound to the mixed immunoaffinity removal medium. The contacting results in a bound fraction (comprising proteins bound to the mixed immunoaffinity removal medium) and an unbound fraction (comprising proteins that do not specifically bind to the mixed immunoaffinity removal medium and are recovered, e.g. in a flowthrough fraction). The unbound fraction may be recovered using any effective method, for example, if the mixed immunoaffinity removal medium is in a ‘spin column’ format, the spin column containing the mixed immunoaffinity removal medium and the sample may be spun in a centrifuge to recover the unbound fraction. The spin column may be washed with a wash buffer to rinse off further unbound protein. The bound fraction may then be eluted using, e.g. a shift in buffer conditions, and then recovered separately. After elution of the bound fraction, it may be further analysed to obtain information about the bound fraction, e.g. to quantitate the serum abundant proteins that were in the original sample but were retained on the mixed immunoaffinity removal medium.

The ceruloplasmin does not substantially bind to the multiple immunoaffinity removal medium, and, as such, is recovered in the unbound fraction. In particular embodiments, at least about 30% of the ceruloplasmin is recovered in the flowthrough fraction, e.g. at least about 40%, at least about 50%, at least about 60%, at least about 70%, at least about 80%, or at least about 90% (or more) of the ceruloplasmin is recovered in the flowthrough fraction. The percentage recovery may be established by spiking a ceruloplasmin-depleted sample with a known quantity of ceruloplasmin to provide a spiked-in sample, then contacting the spiked-in sample with the multiple immunoaffinity removal medium, recovering an unbound protein fraction (e.g. a flowthrough fraction), and then quantitating the ceruloplasmin recovered in the unbound protein fraction. The ceruloplasmin-depleted sample may be obtained using any suitable method, e.g. first contacting a sample with an immunoaffinity medium which specifically binds to ceruloplasmin and allows other proteins to be recovered to provide the ceruloplasmin-depleted sample. The conditions for contacting the spiked-in sample with the multiple immunoaffinity removal medium will typically be essentially the same as used with experimental samples (i.e. samples to be analyzed according to the present method).

Serum fractionation using a mixed immunoaffinity removal medium removes the serum abundant proteins prior to subjecting the unbound fraction to the separation method and analysis by ICP-MS. In certain embodiments, the proteins in the unbound fraction may be preconcentrated (e.g., several unbound fractions of the same sample can be combined after depletion and further concentrated) prior to subjecting the unbound fraction to the separation method.

After recovery of the unbound fraction from the multiple immunoaffinity removal medium, the unbound fraction is subjected to a separation method. The separation method may be any separation technique known in the biochemical arts which results in the separation of protein components in a mixture comprising the protein components and from which a fraction or aliquot comprising a purified protein component may be obtained. Separation methods which may be employed include one or more of the following: high performance liquid chromatography (HPLC), size exclusion chromatography (SEC), gel filtration, reverse phase (RP) chromatography, ion exchange chromatography, hydrophobic interaction chromatography (HIC), other liquid chromatography methods, capillary electrophoresis (CE), polyacrylamide gel electrophoresis (PAGE). In particular embodiments, the separation method is a column chromatography method that provides an outflow that is monitored by a detector, e.g. an absorbance detector, typically a UV absorbance detector or other detector. In typical embodiments, the separation method provides an interface for sampling the outflow and transferring a portion of the outflow to an ICP-MS apparatus for analysis. For example, the separation method may employ a column which is operably coupled to an autosampler for withdrawing aliquots from the outflow of the columns and delivering the aliquots to an ICP-MS apparatus for analysis.

In typical embodiments, the separation method is a column chromatography method that includes a column having an outflow and a detector (e.g. a UV absorbance detector or other detector) operably coupled to the outflow. The detector allows the collection of data from the separation method that is independent of the copper ion specific signal obtained from the ICP-MS analysis. The data from the separation method may then be correlated with the copper ion specific signal obtained from the ICP-MS analysis.

In particular embodiments, a continuous stream of fluid from the outflow of a chromatography column used in the separation method may be transferred to the ICP-MS apparatus, and the continuous stream may be sampled for ICP-MS analysis. In certain embodiments, individual aliquots are periodically obtained from the outflow, and the individual aliquots are transferred to the ICP-MS apparatus for analysis. In some embodiments, the aliquots may be subjected to further processing prior to the ICP-MA analysis.

In typical embodiments, an aliquot containing ceruloplasmin (e.g. at least a portion of the column outflow mentioned above) is analyzed using inductively coupled plasma-mass spectrometry (ICP-MS). The ICP-MS provides a copper ion specific signal. As used herein, an “ion specific signal” is the signal obtained for an ion at a specific mass to charge ratio, as determined by ICP-MS analysis. The present method includes obtaining a copper ion specific signal by analyzing the separated unbound fraction. In addition to the copper ion specific signal, in particular embodiments, the method includes obtaining at least one additional ion specific signal by analyzing the separated unbound fraction. The at least one additional ion specific signal may be, e.g., an iron ion specific signal, a zinc ion specific signal, a calcium ion specific signal, a cobalt ion specific signal, other desired ion specific signal. ICP-MS allows several elements to be identified simultaneously by scanning the mass analyzer, thereby providing ion specific signals for a desired set of ions, such as the copper, iron, zinc, calcium, cobalt, or other desired ions.

The present method includes evaluating the sample for ceruloplasmin. The evaluation may include determining the amount of ceruloplasmin, as well as determining whether it is present or absent. The amount may be an absolute or relative amount. The evaluation may also include determining the amount of another material (including a determination of the presence of absence of the other material). Evaluating the sample may include comparing the copper ion specific signal with a standard curve. In certain embodiments, the method includes establishing a standard curve to reference when comparing with the copper ion specific signal from analysis of a sample. In addition, as mentioned herein, data from the separation method obtained using a detector employed as part of the separation method may be correlated with the copper ion specific signal obtained from the ICP-MS analysis. In an embodiment, the unbound fraction is subjected to the separation method and data is collected from the outflow of the separation method to give a separation profile (e.g. an elution profile). Then a portion of the separation profile is integrated to quantitate the ceruloplasmin, wherein the portion of the separation profile integrated is determined by using the copper ion specific signal to indicate the portion of the separation profile which includes the elution of ceruloplasmin.

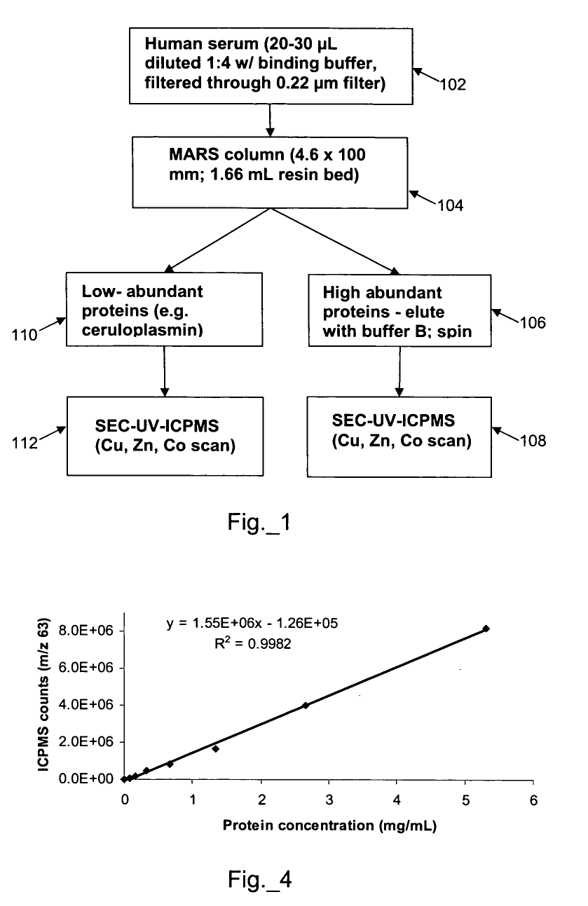

Referring now to FIG. 1, an embodiment of a method of evaluating a sample for ceruloplasmin is charted out. At 102, a serum sample is obtained and prepared for analysis by dilution and filtration through a 0.22 μm filter. At 104, the prepared sample is then contacted with a MARS column (commercially available from Agilent Technologies, Palo Alto, Calif.). An unbound fraction is recovered (at 110) from the MARS column. The unbound fraction includes proteins, including ceruloplasmin, that are not retained by the multiple immunoaffinity removal medium of the MARS column. The unbound fraction (recovered as a flowthrough fraction) is then analysed (at 112) using size exclusion chromatography-HPLC (SEC-HPLC) coupled with a UV detector and an ICP-MS analysis system. Optionally, the bound fraction, including the serum abundant proteins which are retained by the multiple immunoaffinity removal medium of the MARS column, is eluted from the MARS column and recovered separately from the unbound fraction (at 106). The recovered bound fraction is then subjected to analysis (at 108) using size exclusion chromatography-HPLC (SEC-HPLC) operably coupled with a UV detector and an ICP-MS analysis system.

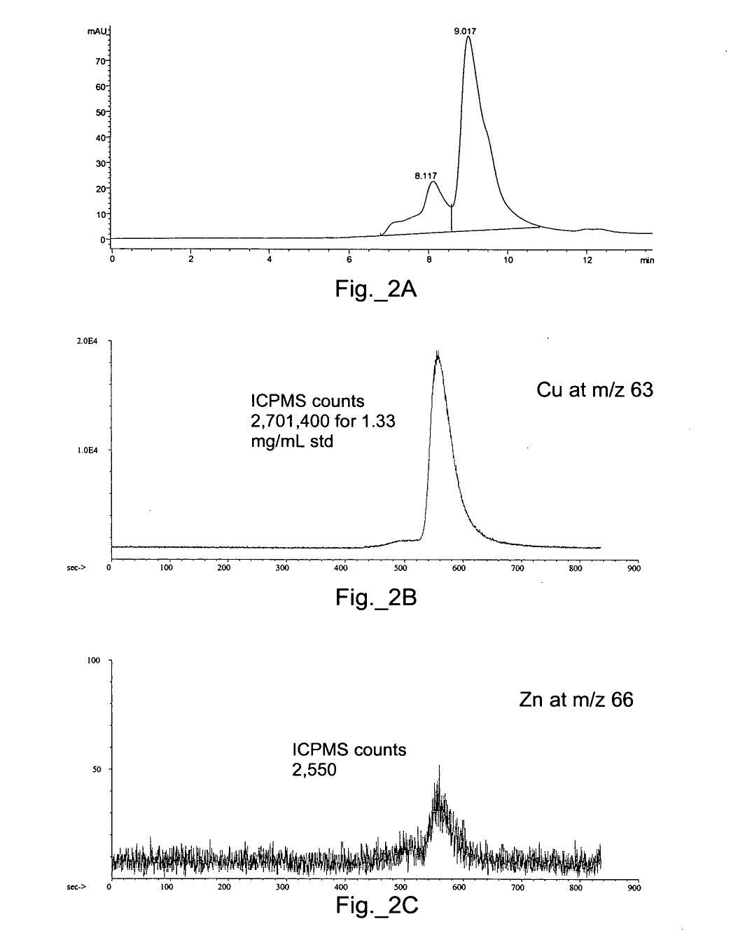

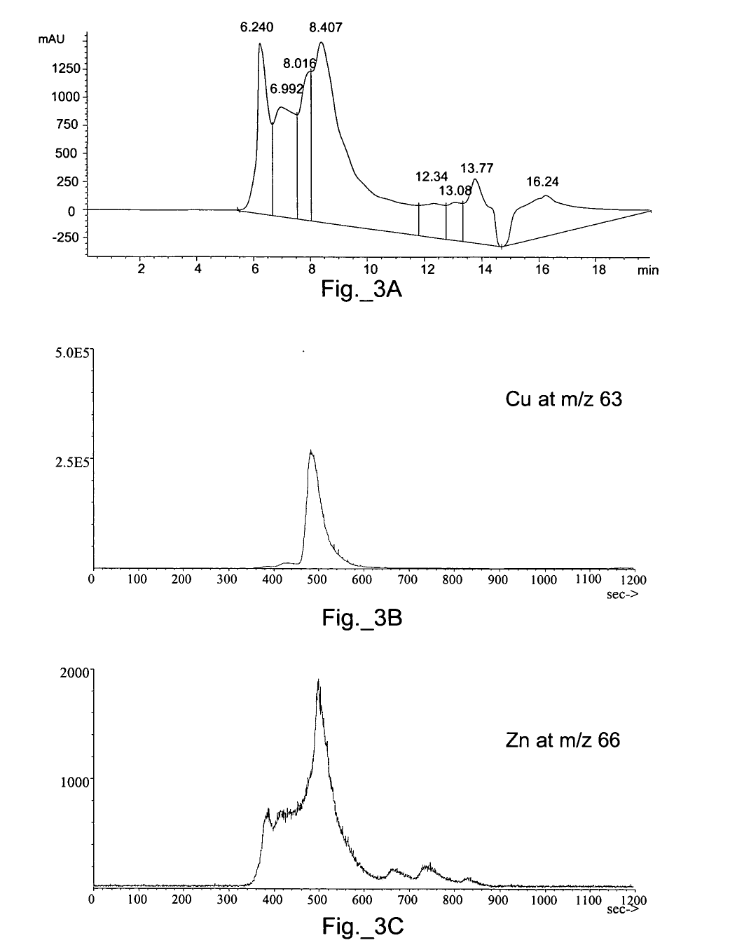

In particular embodiments, the analyses conducted using SEC-HPLC operably coupled with the UV detector and an ICP-MS analysis system provides data that may be subjected to further analysis to determine the presence of and/or the amount of ceruloplasmin in the sample analyzed. The data may be plotted to provide elution profiles, such as those shown in FIGS. 2A-2C and in FIGS. 3A-3C. FIG. 2A shows the elution profile for a purified ceruloplasmin standard solution analyzed as described herein. The elution profile is a plot of the UV absorbance as a function of elution time from the SEC-HPLC. FIG. 2B and FIG. 2C show the copper ion specific signal and the zinc ion specific signal, respectively, obtained from the ICP-MS as a function of elution time from the SEC-HPLC.

FIG. 4 shows a standard curve obtained by assaying a series of solutions having known concentrations of ceruloplasmin according to the method described herein. Seven solutions with concentrations ranging from 0.0645 μg/ml to 5.32 μg/ml, plus a blank (zero) solution were analyzed as described and the data plotted in FIG. 4. Such standard curves typically are determined under experimental conditions that closely correspond to the conditions used for analyzing an experimental sample (e.g. a sample to be analyzed according to the methods described herein). Results obtained from the experimental sample are then related to the standard curve to provide an estimate of the concentration of ceruloplasmin in the experimental sample. Selection of experimental conditions typically depends on such factors as sample source, sample preparation, flow rates in the separation method, standard protocols, equipment selection, etc., and as such will vary. Experimental conditions that provide acceptable results may be established through routine experimentation.

A standard curve may be established under a desired set of experimental conditions and then used in the evaluation of experimental samples using the desired set of experimental conditions. As an example, FIGS. 3A-3C show elution profiles for a serum sample analyzed as described herein. The elution profile of FIG. 3A is a plot of the UV absorbance as a function of elution time from the SEC-HPLC. FIG. 3B and FIG. 3C show the copper ion specific signal and the zinc ion specific signal, respectively, obtained from the ICP-MS as a function of elution time from the SEC-HPLC. The data obtained from the analysis of the experimental sample (e.g. the serum sample) may then be compared with a standard curve such as the one shown in FIG. 4 to obtain a value for ceruloplasmin concentration in the experimental sample. In addition, the presence of the copper ion specific signal obtained during analysis of an experimental sample may be correlated with the elution time of the ceruloplasmin standard to confirm that the species eluted at that elution time is ceruloplasmin.

The present invention provides a useful analytical tool for detecting ceruloplasmin in human serum. The ICP ion source gives primarily monoatomic ions for most elements, which allows them to be identified from their mass spectra. Furthermore, ICP-MS allows several elements to be identified simultaneously by scanning the mass analyzer, has excellent sensitivity, and when used with enriched stable isotopes it allows speciation work to be performed in one experiment. Results indicate that a Cu scan (m/z 63) by ICP-MS can be used as an indicator of the presence of ceruloplasmin in the sample. A review of ICP-MS may be found in: Alan Newman, Analytical Chemistry (1996) 68: 46A-51A.

### EXPERIMENTAL

Protein separation was achieved on a silica TSKGe1 column SW3000 (30 cm×4.6 mm inner diameter with a particle size of 4 μm and pore size of 25 nm) from Tosoh Bioscience. Analyses were conducted on an Agilent Technologies 1100 Series High Performance Liquid Chromatography system. A 0.1M Tris mobile phase was used at a flow rate of 0.3 mL/min (several pHs were investigated). Speciation was carried out on an Agilent Technologies 7500 cs ICP-MS with a quadrupole mass analyzer and an Octopole Reaction System (ORS) for matrix-based interference removal. Helium was used as the reaction gas at 3.5 mL/min.

Serum samples were analyzed before and after removal of six most abundant proteins (i.e., albumin, IgG, IgA, transferrin, haptoglobin and anti-trypsin). The removal of the high-abundant proteins was performed with the Agilent Multiple Affinity Removal System (MARS) column.

QC procedures included column performance verification using a protein ladder (5 proteins), method blanks, analysis of a certified serum sample (Seronorm 1), and analysis of spiked samples.

The practice of the present invention will employ, unless otherwise indicated, conventional techniques of synthetic organic chemistry, biochemistry, molecular biology, and the like, which are within the skill of the art. Such techniques are explained fully in the literature. This description puts forth how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. Standard temperature and pressure are defined as 20° C. and 1 atmosphere.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that if there is a conflict in definitions the definitions explicitly set forth herein shall control.

What is claimed is:

1. A method of evaluating a sample for ceruloplasmin, the sample comprising serum-abundant proteins and ceruloplasmin, the method comprising:
contacting the sample with a multiple immunoaffinity removal medium under conditions sufficient to provide for binding of the serum-abundant proteins to the multiple immunoaffinity removal medium to result in an unbound fraction comprising the ceruloplasmin;
subjecting the unbound fraction to a separation method to provide an aliquot comprising purified ceruloplasmin;
analyzing the aliquot by ICP-MS to provide a copper ion specific signal; and
evaluating the sample for ceruloplasmin based on the copper ion specific signal.

2. The method of claim 1, wherein the multiple immunoaffinity removal medium specifically binds to at least three proteins selected from the group consisting of albumin, IgG, IgM, IgA, fibrinogen, alpha-2 macroglobulin, transferrin, alpha-1 antitrypsin, haptoglobin, alpha-1 acid glycoprotein, apolipoprotein A-I, and apolipoprotein A-II.

3. The method of claim 1, wherein the multiple immunoaffinity removal medium specifically binds to at least five proteins selected from the group consisting of albumin, IgG, IgM, IgA, fibrinogen, alpha-2 macroglobulin, transferrin, alpha-1 antitrypsin, haptoglobin, alpha-1 acid glycoprotein, apolipoprotein A-I, and apolipoprotein A-II.

4. The method of claim 1, wherein the multiple immunoaffinity removal medium is employed in a spin column format.

5. The method of claim 1, further comprising, after contacting the sample with the multiple immunoaffinity removal medium, recovering the unbound fraction and concentrating the unbound fraction prior to subjecting the unbound fraction to the separation method.

6. The method of claim 1, wherein at least about 30% of the ceruloplasmin is recovered in the unbound fraction.

7. The method of claim 1, wherein at least about 50% of the ceruloplasmin is recovered in the unbound fraction.

8. The method of claim 1, wherein at least about 70% of the ceruloplasmin is recovered in the unbound fraction.

9. The method of claim 1, further comprising eluting the serum abundant proteins bound to the multiple immunoaffinity removal medium to result in an eluant fraction comprising the serum abundant proteins, and then subjecting the eluant fraction to further analysis by ICP-MS.

10. The method of claim 1, wherein the separation method comprises a liquid chromatography method selected from the group consisting of high performance liquid chromatography (HPLC), size exclusion chromatography (SEC), gel filtration, reverse phase (RP) chromatography, ion exchange chromatography, and hydrophobic interaction chromatography (HIC).

11. The method of claim 1, wherein the separation method comprises a liquid chromatography method selected from the group consisting of size exclusion chromatography (SEC), gel filtration, reverse phase (RP) chromatography, ion exchange chromatography, and hydrophobic interaction chromatography (HIC).

12. The method of claim 1, wherein the separation method comprises size exclusion chromatography (SEC).

13. The method of claim 1, wherein the separation method includes a column having an outflow which is operably coupled to an autosampler, the autosampler operable to withdraw aliquots from the outflow and deliver the aliquots to an ICP-MS apparatus for analysis.

14. The method of claim 1, further comprising, prior to evaluating the sample for ceruloplasmin based on the copper ion specific signal, establishing a standard curve using a set of control samples having known concentrations of ceruloplasmin.

15. The method of claim 1, wherein evaluating the sample for ceruloplasmin based on the copper ion specific signal includes referencing a standard curve to determine ceruloplasmin concentration.

16. The method of claim 1, wherein analyzing the aliquot by ICP-MS provides at least one additional ion specific signal.

17. The method of claim 1, wherein analyzing the aliquot by ICP-MS provides at least one additional ion specific signal, the at least one additional ion specific signal selected from an iron ion specific signal, a zinc ion specific signal, a calcium ion specific signal, and a cobalt ion specific signal.

18. The method of claim 1, wherein the separation method includes a column having an outflow and a detector operably coupled to the outflow to obtain data from the separation method that is independent of the copper ion specific signal, and correlating the data from the separation method with the copper ion specific signal.
